SEQUENCE LISTING (8 sequences)

SEQ ID NO: 1 - DNA, 300 bp, Saccharomyces sp.
atgcaattctctactgtcgtttccgtcgctttcgtcgccttggccaactttgttgccgctgaatctgctgccgccatctctcaaattactgatggtcaaatccaagctaccactgctaccaccactgctccatccaccactgccgctccatccaccactgctgctccatcttcttcccacgttgaaaccgtttctgcttccagcactgaaaccatttctcaacaaactgaaaatggtgctgctaaggccgctgtcggtatgggtgccggtgctctagctgccgctgctatgttgttgtaa

SEQ ID NO: 2 - protein, 99 aa, Saccharomyces sp.
MQFSTVVSVAFVALANFVAAESAAAISQITDGQIQATTATTTAPSTTAAPSTTAAPSSSHVETVSASSTETISQQTENGAAKAAVGMGAGALAAAAMLL

SEQ ID NO: 3 - DNA, 279 bp, Saccharomyces sp.
atgcaattctctactgtcgcttccgttgctttcgtcgctttggctaactttgttgccgctgaatccgctgccgccatttctcaaatcactgacggtcaaatccaagctactaccactgctaccaccgaagctaccaccactgctgccccatcttccaccgttgaaactgtttctccatccagcaccgaaactatctctcaacaaactgaaaatggtgctgctaaggccgctgtcggtatgggtgccggtgctctagctgctgctgctatgttgttataa

SEQ ID NO: 4 - protein, 92 aa, Saccharomyces sp.
MQFSTVASVAFVALANFVAAESAAAISQITDGQIQATTTATTEATTTAAPSSTVETVSPSSTETISQQTENGAAKAAVGMGAGALAAAAMLL

SEQ ID NO: 5 - DNA, 40 bp, Artificial Sequence
  Primer
gagctcatagcggccatgcaattctctactgtcgtttccg

SEQ ID NO: 6 - DNA, 42 bp, Artificial Sequence
  Primer
ggatcctatgcggccgcaggtaaaaaggaatcaaacacttac

SEQ ID NO: 7 - DNA, 36 bp, Artificial Sequence
  Primer
gagctcatgcaattctctactgtcgcttccgttgct

SEQ ID NO: 8 - DNA, 36 bp, Artificial Sequence
  Primer
ggatcccattcgaagagaaatcacaggaacatcgtt

(19) United States
(12) Patent Application Publication
(10) Pub. No.: US 2009/0274794 A1
(43) Pub. Date: Nov. 5, 2009

(54) Gene Encoding Cell Wall Mannoprotein and use Thererof

(75) Inventors: Yoshihiro Nakao, Osaka (JP); Yukiko Kodama, Osaka (JP); Tomoko Shimonaga, Osaka (JP); Fumihiko Ohmura, Osaka (JP)

Correspondence Address: DRINKER BIDDLE & REATH (DC), 1500 K STREET, N.W., SUITE 1100, WASHINGTON, DC, 20005-1209, US

(73) Assignee: SUNTORY LIMITED, OSAKA (JP)

(21) Appl. No.: 11/991,797

(22) PCT Filed: Sep. 21, 2006
(86) PCT No.: PCT/JP2006/319228

§ 371 Date: Mar. 11, 2008

(30) Foreign Application Priority Data
Sep. 22, 2005 (JP) 2005-276423
Dec. 27, 2005 (JP) 2005-375016

(51) Int. Cl. C12N 1/19; C07H 21/04; C07K 14/00; C12N 15/63; C12Q 1/68; C12C 11/09; A23L 1/185; C12G 1/073; C12H 1/14

(52) U.S. Cl. 426/15; 536/23.1; 530/350; 435/320.1; 435/254.2; 435/6; 426/11; 426/16; 426/592

(57) ABSTRACT

The present invention relates to a brewer's yeast which produces alcoholic beverages having an ability for reducing the haze level, alcoholic beverages produced using such a yeast, and a method of producing such alcoholic beverages. More specifically, the present invention relates to a yeast which can reduce the level of haze in the product by increasing the level of expression of ScCWP2 gene encoding cell wall mannoprotein Cwp2p in brewer's yeast, or non-ScCWP2 gene characteristic to beer yeast, and to a method of producing alcoholic beverages using such a yeast.

### TECHNICAL FIELD

The present invention relates to a gene encoding a cell wall mannoprotein and uses thereof. The present invention relates in particular to a brewer's, yeast which produces alcoholic beverages having an ability for reducing the haze level, alcoholic beverages produced using such a yeast, and a method of producing such alcoholic beverages. More specifically, the present invention relates to a yeast which can reduce the level of haze in the product by increasing the level of expression of ScCWP2 gene encoding cell wall mannoprotein Cwp2p in brewer's yeast, or non-ScCWP2 gene characteristic to beer yeast, and to a method of producing beverages using such a yeast.

### BACKGROUND ART

Liquors include, for example, fermented liquors made by alcohol fermentation with yeasts or the like, using sugars and starchy material as starting materials, such as wine, beer, sake and the like.

For example, beer is produced by obtaining wort by saccharization using malt as a major material, subjecting the wort to main fermentation using yeast, and subjecting the fermented wort to post fermentation (aging) followed by filtration and bottling. It is a quite important demand for the quality of the alcoholic beverages made by fermentation (particularly pale-colored beverages) such as beer thus produced that mere is no turbidity in the period from production to consumption, or the alcoholic beverage is stable against turbidity.

The cause of turbidity of beer is roughly divided into biological turbidity and non-biological turbidity. Biological turbidity is caused by contamination with microorganisms. Non-biological turbidity is ascribed to denaturation of beer's own component, for example formation of protein components collectively referred to a haze-forming protein formed by association of the protein components and polyphenol (K. Asano et al., ASBC Journal, 40:147-154, 1982; J. A. Delcour et al., MBAA Technical Quarterly, 25:62-66, 1988). Commonly formed turbidity is non-biological turbidity. While causative substances and mechanisms of non-biological turbidity have not been elucidated yet, it has been supposed in recent years that malt, protein components originating from hop and polyphenols bind to cell wall components (mannoprotein and the like) originating from yeast to gradually form large particles.

Recently, a relation between the degree of exfoliation of mannoprotein from yeast and the level of haze has been found (F. Omura et al., 30^{th} EBC Congress, SUMMARIES PRESENTATION, 19, 2005). Cwp2p is one of major mannoproteins constituting the cell wall, and serves for stabilization of the cell wall arid-resistance at low pH (M Skrzypek et. al, Curr Genet, 38,191-201,2000).

### DISCLOSURE OF INVENTION

As noted above, while the causative substances of non-biological turbidity and the mechanism for forming non-biological turbidity have not been elucidated yet, it has been urgently desired for controlling the quality of the fermented alcoholic beverage to reduce such non-biological turbidity.

The present inventors made, intensive studies to solve the above problems, and as a result succeeded in identifying and isolating a gene encoding a cell wall mannoprotein from lager brewing yeast. Moreover, a transformed yeast wherein the obtained gene was introduced and expressed, was prepared, to confirm that the amount of haze produced was reduced, thereby completing the present invention.

Thus, the present invention relates to a cell Avail mannoprotein gene existing specifically in a lager brewing yeast, to a protein encoded by said, gene, to a transformed yeast in which the expression of said gene is controlled, to a method for controlling the amount of haze in a product by using a yeast in which the expression of said, gene is controlled. More specifically, the present invention provides the following polynucleotides, a vector comprising said polynucleotide, a transformed yeast introduced with said vector, a method for producing alcoholic-beverages by using said transformed yeast, and the like.

(1) A polynucleotide selected from the group consisting of:

(a) a polynucleotide comprising a polynucleotide consisting of the nucleotide sequence of SEQ ID NO. 1;

(b) a polynucleotide comprising a polynucleotide encoding a protein consisting of the amino acid sequence of SEQ ID NO: 2;

(c) a polynucleotide comprising a polynucleotide encoding a protein consisting of the amino acid sequence of SEQ ID NO:2 with one or more amino acids thereof being deleted, substituted, inserted and/or added, and functioning as a cell wall mannoprotein;

(d) a polynucleotide comprising a polynucleotide encoding a protein having an amino acid sequence having 60% or higher identity with the amino acid, sequence of SEQ ID NO:2, and functioning as a cell wall mannoprotein;

(e) a polynucleotide comprising a polynucleotide which hybridizes to a polynucleotide consisting Of a nucleotide sequence complementary to the nucleotide sequence of SEQ ID NO: 1 under stringent conditions, and which encodes a protein functioning as a cell wall mannoprotein; and

(f) a polynucleotide comprising a polynucleotide which hybridizes to a polynucleotide consisting of a nucleotide sequence complementary to the nucleotide sequence of the polynucleotide encoding the protein of the amino acid sequence of SEQ ID NO: 2 under stringent conditions, and which encodes a protein functioning as a cell wall mannoprotein,

(2) The polynucleotide of (1) above selected from the group consisting of:

(g) a polynucleotide comprising a polynucleotide encoding a protein consisting of the amino acid sequence of SEQ ID NO: 2, or encoding an amino acid sequence of SEQ ID NO: 2 wherein 1 to 10 amino acids thereof is deleted, substituted, inserted and/or added, and wherein said protein functions as a cell wall mannoprotein;

(h) a polynucleotide comprising a polynucleotide encoding a protein having 90% or higher identity with the amino acid sequence of SEQ ID NO: 2, and functioning as a cell wall mannoprotein; and

(i) a polynucleotide comprising a polynucleotide which hybridizes to SEQ ID NO: 1 or which hybridizes to a nucleotide sequence complementary to the nucleotide sequence of SEQ ID NO: 1 under stringent conditions, and, which encodes a protein functioning as a cell wall mannoprotein.

(3) The polynucleotide of (1) above comprising a polynucleotide consisting of SEQ ID NO: 1.

(4) The polynucleotide of (1) above comprising a polynucleotide encoding a protein consisting of SEQ ID NO: 2.

(5) The polynucleotide of any one of (1) to (4) above, wherein the polynucleotide is DNA.

(6) A protein encoded by the polynucleotide of any one of (1) to (5) above.

(7) A vector comprising the polynucleotide of any one of (1) to (5) above.

(7a) The vector of (7) above, which comprises the expression cassette comprising fee following components:

(x) a promoter that can be transcribed in a yeast cell;

(y) the polynucleotides described in (1) to (5) above linked to the promoter in a sense direction; and

(z) a signal mat can function in a yeast with respect to transcription termination and polyadenylation of a RNA molecule.

(8) A vector comprising the polynucleotide selected from the group consisting of:

(j) a polynucleotide comprising a polynucleotide comprising a polynucleotide encoding a protein consisting of the amino acid sequence of SEQ ED NO: 4, or encoding an amino acid sequence of SEQ ID NO: 4 wherein 1 to 10 amino acids thereof is deleted, substituted, inserted, and/or added, and wherein said protein functions as a cell wall mannoprotein;

(k) a polynucleotide comprising a polynucleotide comprising a polynucleotide encoding a protein having 90% or higher identity, with the amino acid sequence of SEQ ID NO: 4, and functioning as a cell wall mannoprotein; and

(l) a polynucleotide comprising a polynucleotide comprising a polynucleotide which hybridizes to SEQ ID NO: 3 or which hybridizes, to a nucleotide sequence complementary to the nucleotide sequence of SEQ ID NO: 3 under stringent conditions, and which encodes a protein functioning as a cell wall mannoprotein.

(9) A yeast, wherein the vector of (7) or (8) above is introduced,

(10) The yeast of (9) above, wherein hydrogen sulfide-producing ability is reduced by introducing the vector of (7) or (8) above.

(11) The yeast of (10) above, wherein a haze-producing ability is reduced by increasing an expression level of the protein of (6) above.

(12) A method for producing an alcoholic liquor by using the yeast of any one of (9) through (11) above.

(13) The method for producing an alcoholic liquor of (12) above, wherein the brew is a malt liquor.

(14) The method for producing an alcoholic liquor of (12) above, wherein the brew is a wine.

(15) An alcoholic liquor, which is produced by the method of any one of (12) through (14) above.

(16) A method for assessing a test yeast for its haze-producing ability, comprising using a primer or a probe designed based on a nucleotide sequence of a gene encoding a cell wall mannoprotein having the nucleotide sequence of SEQ ID NO: 1 or SEQ ID NO: 3.

(16a) A method for selecting a yeast having a low haze-producing ability by using the method in (16) above.

(16b) A method for producing an alcoholic liquor (for example, beer) by using the yeast selected with the method in (16a) above.

(17) A method for assessing a test yeast for its haze-producing capability, comprising: culturing a test yeast; and measuring an expression level of a gene encoding a cell wall mannoprotein having the nucleotide sequence of SEQ ID NO: 1 or SEQ ID NO: 3.

(18) A method for selecting a yeast, comprising: culturing test yeasts; quantifying the protein of (6) above of measuring an expression level of a gene encoding a cell wall mannoprotein having the nucleotide sequence of SEQ ID NO: 1 or SEQ ID NO: 3; and selecting a test yeast haying said protein amount or said gene expression level according to a target haze-producing capability.

(18a) A method for selecting a yeast, comprising: culturing test yeasts; measuring haze-producing capability; and selecting a test yeast having a target haze-producing capability.

(19) The method for selecting a yeast of (18) above, comprising: culturing a reference yeast and test yeasts; measuring an expression level of a gene encoding a cell wall mannoprotein having the nucleotide sequence of SEQ ID NO: 1. For SEQ ID NO: 3 in each yeast; and Selecting a test yeast having the gene expressed higher than that in the reference yeast

(20) The method for selecting a yeast of (18) above-comprising: culturing a reference yeast and test yeasts; quantifying the protein of (6) above in each yeast; and selecting a test yeast having said protein for a larger amount than that in the reference yeast That is, the method for selecting a yeast of (18) above comprising: culturing plural yeasts; quantifying the protein of (6) above in each yeast; and selecting a test yeast having a large amount of the protein from them.

(21) A method for producing an alcoholic beverage comprising: conducting fermentation for producing an alcoholic beverage using the yeast according to any one of (9) to (11) or a yeast selected by the method according to any one of (18) to (20); and adjusting the production amount of haze.

According to the method for producing alcoholic beverages by using the transformed yeast, since the cell wall structure of the yeast can be stabilized, it is possible to produce alcoholic beverages wherein the haze level can be lowered in beer fermentation and the finished product.

### BRIEF DESCRIPTION OF DRAWINGS

FIG. 1 shows the cell growth with time upon beer fermentation test in Example 2. The horizontal axis represents fomentation time while the vertical axis represents optical density at 660 nm (OD660).

FIG. 2 shows the sugar consumption with time upon beer fermentation test in Example 2. The horizontal axis represents fermentation time while the vertical axis represents apparent extract concentration (w/w %).

FIG. 3 shows the expression profile of non-ScCWP2 gene in yeasts upon beer. fermentation test in Example 2. The horizontal axis represents fermentation time while the vertical axis represents the intensity of detected signal.

FIG. 4 shows the cell growth with time upon fermentation test in this Example. The horizontal axis represents fermentation time while the vertical axis represents optical density at 660 nm (OD660).

FIG. 5 shows the sugar assumption with time upon beer fermentation test in this Example. The horizontal axis represents fermentation time while the vertical axis represents apparent extract concentration (w/w %).

FIG. 6 shows the expression profile of ScCWP2 gene in yeasts upon beer fermentation test in Example 6. The horizontal axis represents fermentation time while the vertical axis represents the intensity of detected signal

FIG. 7 shows the cell growth with time upon fermentation test in this Example. The horizontal axis represents fermentation time while the vertical axis represents optical density at 660 nm (OD660).

FIG. 8 shows the sugar consumption with time upon beer fermentation test in this Example. The horizontal axis represents fermentation time while the vertical axis represents apparent extract concentration (w/w %).

### BEST MODES FOR CARRYING OUT THE INVENTION

The present inventors conceived that it is possible to stabilize a cell wall of the yeast by increasing a cell wall mannoprotein of the yeast The present inventors have studied based on this conception and as a result, isolated and identified non-ScCWP2 gene encoding a cell wall mannoprotein unique to lager brewing yeast based oh the lager brewing yeast genome information mapped according to the method disclosed in Japanese Patent Application Laid-Open No. 2004-283169. The nucleotide sequence of the gene arid an amino acid sequence of a protein . encoded by the gene are represented by SEQ ID NO: 1 and SEQ ID NO: 2, respectively. The present inventors isolated and identified ScCWP2 gene encoding a cell wall mannoprotein unique to lager brewing yeast based on the lager brewing yeast genome information mapped according to the method disclosed in Japanese Patent Application Laid-Open No. 2004-283169. The nucleotide sequence of the gene and an amino acid sequence of a protein encoded by the gene are represented by SEQ ID NO: 3 and SEQ ID NO: 4, respectively. The sequence information of ScCWP2 may be obtained from the genome database of S. cerevisiae (http://genome-www.stanford.edu/Saccharomyces/).

### 1. Polynucleotide of the Invention

First of all the present invention provides (a) a polynucleotide comprising a polynucleotide of the nucleotide sequence of SEQ ID NO: 1 or SEQ ID NO: 3; and (b) a polynucleotide comprising a polynucleotide encoding a protein of the amino acid sequence of SEQ ID NQ: 2 or SEQ ID NO: 4. The polynucleotide can be DNA or RNA.

The target polynucleotide of the present invention is not limited to the polynucleotide encoding a cell wall mannoprotein derived from lager, brewing yeast and may include other polynucleotides encoding proteins having equivalent functions to said protein. Proteins with equivalent functions include, for example, (c) a protein of an amino acid sequence of SEQ ID NO: 2 or SEQ ID NO: 4 with one or more amino acids thereof being deleted, substituted, inserted and/or added and functioning as a cell wall mannoprotein (a mannoprotein composing a cell wall).

Such proteins include a protein consisting of an amino acid sequence of SEQ ID NO: 2 or SEQ ID NO: 4 with, for example, 1 to 100, 1 to 90, 1 to 80, 1 to 70, 1 to 60, 1 to 50, 1 to 40, 1 to 39, 1 to 38, 1 to 37, 1 to 36, 1 to 35, 1 to 34, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20; 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6 (1 to several amino acids), 1 to 5, 1 to 4, 1 to 3, 1 to 2, or 1 amino acid, residues thereof being deleted, substituted, inserted and/or added arid functioning as a cell wall mannoprotein. In general, the number of deletions, substitutions, insertions, and/or additions is preferably smaller. In addition, such proteins include (d) a protein having an amino acid sequence with about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, .93% or higher, 94% or higher, 95% or higher, 96% or higher, 97% or higher, 98% or higher, 99% or higher, 99.1% or higher, 99.2% or higher, 993% or higher, 99.4% or higher, 99.5% or higher, 99.6% or higher, 99.7% or higher, 99.8% or higher, or 99.9% or higher identity with the amino acid sequence of SEQ ID NO: 2 or SEQ ID NO: 4, and functioning as a cell wall mannoprotein. In general the percentage identity is preferably higher.

Whether a certain protein functions as a cell wall mannoprotein or not, can be judged by, for example, separating a sample by SDS electrophoresis according to the molecular weight, and subjecting the protein separated from the sample to a technique called affinoblotting detection utilizing a lectine, Concanavalin A that recognizes a mannose site of mannoprotein and binds thereto (Faye L and Chrispeels M J, Anal Biochem, 1985).

Furthermore, the present invention also contemplates (e) a polynucleotide comprising a polynucleotide which hybridizes to a polynucleotide consisting of a nucleotide sequence complementary to the nucleotide sequence of SEQ ID NO: 1 or SEQ ID NO: 3 under stringent conditions and which encodes a protein functioning as a cell wall mannoprotein; and (f) a polynucleotide comprising a polynucleotide which hybridizes to a polynucleotide complementary to a nucleotide sequence of encoding a protein of SEQ ID NO: 2 or SEQ ID NO: 4 under stringent conditions, and which encodes a protein functioning as a cell wall mannoprotein.

Herein, “a polynucleotide that hybridizes under stringent conditions” refers to nucleotide sequence, such as a DNA, obtained by a colony hybridization technique, a plaque hybridization technique, a southern hybridization technique or the like using all or part of polynucleotide of a nucleotide sequence complementary to the nucleotide sequence of SEQ ID NO: 1 or SEQ ID NO: 3 or polynucleotide encoding the amino acid sequence of SEQ ID NO: 2 or SEQ ID NO: 4 as a probe. The hybridization method may be a method described, for example, in MOLECULAR Cloning 3rd Ed., CURRENT PROTOCOLS IN MOLECULAR BIOLOGY, John Wiley & Sons 1978-1997.

The term “stringent conditions” as used herein may be any of low stringency conditions, moderate stringency conditions or high stringency conditions. “Low stringency conditions” are, for example, 5×SSC, 5×Denhardts solution, 0.5% SDS, 50% formatmide at 32° C. “Moderate stringency conditions” are, for example, 5×SSC, 5×Denhardt's solution, −0.5% SDS, 50% formamide at 42° C. “High, stringency, conditions” are, for example, 5×SSC, 5×benhardt's solution, 0.5% SDS, 50% formamide at 50° C. Under these conditions, a polynucleotide, such as a DNA, with higher homologyis expected to be obtained efficiently at higher temperature, although multiple factors are involved in hybridization stringency including temperature, probe concentration, probe length, ionic strength, time, salt concentration and others, and one skilled in the art may appropriately select these factors to realize similar stringency.

When a commercially available kit is used for hybridization, for example, Alkphos Direct Labeling Reagents (Amersham Pharmacia) may be used. In this case, according to the attached protocol, after incubation with a labeled probe overnight, the membrane is washed with a primary wash buffer containing 0.1% (w/v) SDS at 55° C., thereby detecting hybridized polynucleotide, such as DNA.

Other polynucleotides that can be hybridized include polynucleotides having about 60% or higher, about 70% or higher, 71% or higher, 72% or higher, 73% or higher, 74% or higher, 75% or higher, 76% or higher, 77% or higher, 78% or higher, 79% or. higher, 80% or higher, 81 % or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 992% or higher, 993% or higher, 99.4% or higher, 99.5% or higher, 99.6% or higher, 99.7% or higher, 99.8% or higher or 99.9% or higher identity to polynucleotide encoding the amino acid sequence of SEQ ID NO: 2 or SEQ ID NO: 4 as calculated by homology search software, such as FASTA and BLAST using default parameters.

Identity between amino acid sequences or nucleotide sequences may be determined using algorithm BLAST by Karim and Altschul (Proc. Natl. Acad. Sci. USA, 87:2264-2268,1990, Proc. Natl. Acad. Sci. USA, 90:5873, 1993). Programs called BLASTN and BLASTX based on BLAST algorithm have been developed (Ahschul S F et al., J. Mol. Biol 215: 403, 1990). When a nucleotide sequence is sequenced using BLASTN, the parameters are, for example, score=100 and word length=12. When an amino acid sequence is sequenced using BLASTX, the parameters are, for example, score=50 and word length=3. When BLAST and Gapped BLAST programs are used, default parameters for each of the programs are employed.

### 2. Protein of the Present Invention

The present invention also provides proteins encoded by any of the polynucleotides (a) to (l) above. A preferred protein of the present invention comprises an amino acid sequence of SEQ ID NO:2 or SEQ ID NO:4 with one or several amino acids thereof being deleted, substituted, inserted and/or added, and functions as a cell wall mannoprotein (which herein may be simply referred to as “a cell wall mannoprotein”).

Such protein includes those having an amino acid sequence of SEQ. ID NO: 2 or SEQ ID NO: 4 with amino acid residues thereof of the number mentioned above being deleted, substituted, inserted and/or added arid functioning as a cell wall mannoprotein. In addition, such protein includes those having homology as described above with the amino acid sequence of SEQ ID NO: 2 or SEQ ID NO: 4 and functioning as a cell wall mannoprotein.

Such proteins may be obtained by employing site-directed mutation described, for example, in MOLECULAR CLONING 3rd Ed., CURRENT PROTOCOLS IN MOLECULAR BIOLOGY, Nuc. Acids. Res., 10:6487 (1982), Proc. Natl. Acad. Sci. USA 79; 6409 (1982), Gene 34:315 (1985), Nuc. Acids. Res., 13:4431 (1985), Proc. Natl. Acad. Sci. USA 82: 488 (1985).

Deletion, substitution, insertion and/or addition of one or more amino acid residues in an amino acid sequence of the protein of the invention means that one or more amino acid residues are deleted, substituted, inserted and/or added at any one or more positions in the same amino acid sequence. Two or more types of deletion, substitution, insertion and/or addition may occur concurrently.

Hereinafter, examples of mutually substitutable amino acid residues are enumerated. Amino acid residues in the same group are mutually substitutable. The groups are provided below.

Group A: leucine, isoleucine, norleucine, valine, norvaline, alanine, 2-aminobutanoic acid, methionine, o-methylserine, t-butylglycine, t-butylalanine, cyclohexylalanine; Group B: asparatic acid, glutamic acid, isoasparatic acid, isoglutamic acid, 2-aminoadipic acid, 2-aminosuberic acid; Group C: asparagine, glutamine; Group D: lysine, arginine, ornithine, 2,4-diaminobutanoic acid, 2,3-diaminopropionic acid; Group E: proline, 3-hydroxyproline, 4-hydroxyproline; Group F: serine, threonine, homoserine; and Group G: phenylalanine, tyrosine.

The protein of the present invention may also be produced by chemical synthesis methods such as Fmoc method (fluorenymethyloxycarbonyl method) and tBoc method (t-butyloxycarbonyl method). In addition, peptide synthesizers available from, for example. Advanced ChemTech, PerkinElmer, Pharmacia, Protein Technology Instrument, Synthecell-Vega, PerSeptive, Shimazu Corp. can also be used for chemical synthesis.

3. Vector of the Invention and Yeast Transformed with the Vector

The present invention then provides a vector comprising the polynucleotide described above. The vector of the present invention is directed to a vector including any of the polynucleotides described in (a) to (l) above. Generally, the vector of the present invention comprises an expression cassette including as components (x) a promoter that can transcribe in a yeast cell; (y) a polynucleotide described in any of (a) to (l) above that is linked to the promoter in sense or antisense direction; and (z) a signal that functions in the yeast with respect to transcription termination and polyadenylation of RNA molecule.

A vector introduced in the yeast may be any of a multicopy type (YEp type), a single copy type (YCp type), or a chromosome integration type (YI type). For example, YEp24 (J. R. Broach et al., Experimental Manipulation of Gene Expression, Academic Press, New York, 83, 1983) is known as a YEp type vector, YCp50 (M. D. Rose et al., Gene 60:237,1987) is known as a YCp type vector, and YIp5 (K. Struhl et al., Proc. Natl. Acad. Sci. USA, 76: 1035,1979) is known as a YIp type vector, all of which are readily available.

Promoters/terminators for adjusting gene expression in yeast may be in any combination as long as they function in the brewery yeast and they are riot influenced by constituents in fermentation broth. For example, a promoter of glyceraldehydes 3-phosphate dehydrogenase gene (TDH3); or a promoter of 3-phosphoglycerate kinase gene (PGK1) may be used These genes have previously been cloned, described in detail, for example, in M. F. Tuite et al., EMBO J., 1,603 (1982), and are readily available by known methods.

Since an auxotrophy marker cannot be used as a selective marker upon transformation for a brewery yeast, for example, a geneticin-resistant gene (G418r), a copper-resistant gene (CUP1) (Marin et al., Proc. Natl. Acad. Sci. USA, 81,337 1984) or a cerulenin-resistant gene (fas2m, PDR4) (Junji Inokoshi et al., Biochemistry, 64, 660, 1992; and Hussain et al., Gene, 101: 149, 1991, respectively) may be used

A vector constructed as described above is introduced into a host yeast Examples of the host yeast include any yeast that can be used for brewing, for example, brewery yeasts for beer, wine and sake. Specifically, yeasts such as genus Saccharomyces may be used . According to the present invention, a lager brewing yeast, for example, Saccharomyces pastorianus W34/70, Saccharomyces carlsbergensis NCYC453 or NCYC456, or Saccharomyces NBRC1951, NBRC1952, NBRC1953 or NBRC1954 may be used. In addition, whisky yeasts such as Saccharomyces cerevisiae NCYC90, wine yeasts such as wine yeasts #1,3 and 4 from the Brewing Society of Japan, and sake yeasts-such as sake yeast #7 and 9 from the Brewing Society of Japan may also be used but hot limited thereto. In the present invention, lager brewing yeasts such as Saccharomyces pastorianus may be used preferably.

A yeast transformation method may be a generally used known method. For example, methods that can be used include but not limited to an electroporation method (Meth. Enzym., 194: 182 (1990)), a spheroplast method (Proc. Natl. Acad. Sci. USA, 75:1929(1978)), a lithium acetate method (J. Bacteriology, 153:163 (1983)), and methods described in Proc Natl. Acad. Sci. USA, 75:1929 (1978); METHODS IN YEAST GENETICS, 2000 Edition: A Cold Spring Harbor Laboratory Course Manual.

More specifically, a host yeast is cultured in a standard yeast nutrition medium (e.g, YEPD medium (Genetic Engineering. Vol. 1,Plenum Press, New York, 117(1979)), etc.) such that OD600 nm will be 1 to 6. This culture yeast is collected by centrifugation, washed and pre-treated with alkali ion metal ion, preferably lithium ion at a concentration of about 1 to 2 M. After the cell is left to stand at about 30° C. for about 60 minutes, if is left to stand with DNA to be introduced (about 1 to 20 μg) at about 30° C. for about another 60 minutes. Polyethyleneglycol, preferably about 4,000 Dalton of polyethyleneglycol, is added to a final concentration of about 20% to 50%. After leaving at about 30° C. for about 30 minutes, the cell is heated at about 42° C. for about 5 minutes. Preferably, this cell suspension is washed with a standard yeast nutrition medium, added to a predetermined amount of fresh standard yeast nutrition medium and left to stand at about 30° C. for about 60 minutes. Thereafter, it is seeded to a standard agar medium containing an antibiotic or the like as a selective marker to obtain a transformant.

Other general cloning techniques may be found, for example, in MOLECULAR CLONING 3rd Ed, and METHODS IN YEAST GENETICS, A LABRATORY MANUAL (Cold Spring Harbor Laboratory Press, Cold Spring Harbor, N.Y.).

### 4. Method of Producing Alcoholic Beverages According to the Present Invention and Alcoholic Beverages Produced by the Method

The vector of the present invention described above is introduced into a yeast suitable for brewing a target alcoholic product This yeast can be used to produce a desired alcoholic beverage with a lowered amount of haze. In addition, yeasts to be selected by the yeast assessment method of the present invention described below can also be used. The target alcoholic beverages include, for example, but riot limited to beer, sparkling liquor (happoushu) such as a beer-taste beverage, wine, sake and the like.

In order to produce these alcoholic beverages, a known technique can be used except that a brewery yeast obtained-according to the present invention is used in the place of a parent strain. Since materials, manufacturing equipment, manufacturing control and the like may be exactly the same as the conventional ones, there is no need of increasing the cost for producing alcoholic beverages with a lowered amount of haze. Thus, according to the present invention, alcoholic beverages with excellent, stability of turbidity and the like can be produced using (he existing facility without increasing the cost

### 5. Yeast Assessment Method of the Invention

The present invention relates to a method for assessing a test yeast for its haze-producing capability by using a primer or a probe designed based on a nucleotide sequence of a gene encoding a cell wall mannoprotein haying the nucleotide sequence of SEQ ID NO:1 or SEQ ID NO:3. General techniques for such assessment method is known and is described in, for example, WO01/040514, Japanese Laid-Open Patent Application No; 8-205900 or the like. This assessment method is described in below.

First, genome of a test yeast is prepared For this preparation, any known method such as Hereford method or potassium acetate method may be used (e.g., Methods in Yeast Genetics, Cold Spring Harbor Laboratory Press, 130 (1990)). Using a primer or a probe designed based on a nucleotide sequence (preferably, ORF sequence) of the gene encoding a cell wall mannoprotein, the. existence of the gene or a sequence specific to the gene is determined in the test yeast genome obtained. The primer or the probe may be designed according to a known technique.

Detection of the gene or the specific sequence may be carried out by employing a known technique. For example, a polynucleotide including part or all of the specific, sequence or a polynucleotide including a nucleotide sequence complementary to said nucleotide sequence is used as one primer, while a polynucleotide including part or all of the sequence upstream or downstream from this sequence or a polynucleotide including a nucleotide sequence complementary to said nucleotide sequence, is used as another primer to amplify a nucleic acid of the yeast by a PCR method, thereby determining the existence of amplified products and molecular weight of the amplified products. The number of bases of polynucleotide used for a primer is generally 10 base pairs (bp) or more, and preferably 15 to 25 bp. In general, the number of bases between the primers is suitably 300 to 2000 bp.

The reaction conditions for PCR are not particularly limited but may be, for example, a denaturation temperature of 90 to 95° C. an annealing temperature of 40 to 60° C., an elongation temperature of 60 to 75° C., and me number of cycle of 10 or more. The resulting reaction product may be separated, for example,, by electrophoresis using agarose, gel to determine the molecular weight of the amplified product This method allows prediction and assessment of the capability of the yeast to produce haze as determined by whether the molecular weight of the amplified product is a size that contains the DNA molecule of the specific part. In addition, by analyzing the nucleotide sequence of the amplified product, the capability may be predicted and/or assessed more precisely.

Moreover, in the present invention, a test yeast is cultured to measure an expression level of the gene encoding a cell wall mannoprotein having the nucleotide sequence of SEQ ID NO: 1 or SEQ ID NO: 3 to assess the test yeast for its haze-producing capability. In measuring an expression level of the gene, the test yeast is cultured and men mRNA or a protein resulting from the gene encoding a cell wall, mannoprotein is quantified. The quantification of mRNA or protein may be carried out by employing a known technique. For example, mRNA may be quantified, by Northern hybridization or quantitative RT-PCR, while protein may be quantified, for example, by Western blotting (CURRENT PROTOCOLS IN MOLECULAR BIOLOGY, John Wiley & Sons 1994-2003). The level of expression of the gene in test yeasts can be predicted by measuring the content of haze in the fermentation liquor obtained when the test yeasts are cultured.

Furthermore, test yeasts are cultured and expression levels of the gene encoding a cell wall mannoprotein having the nucleotide sequence of SEQ ID NO: 1 or SEQ ID NO: 3 are measured to select a test yeast with the gene expression level according to the target capability of producing haze, thereby selecting a yeast favorable for brewing desired alcoholic beverages. In addition, a reference yeast and a test yeast may be cultured so as to measure and compare the expression level of the gene in each of the yeasts, thereby selecting a favorable test yeast. More specifically, for example, a reference yeast and one or more test yeasts are cultured and an expression level of the gene encoding a cell wall mannoprotein having the nucleotide sequence of SEQ ID NO: 1 or SEQ ID NO: 3 is measured in each yeast. By selecting a test yeast with the gene expressed higher than that in the reference yeast, a yeast suitable for brewing alcoholic beverages can selected

Alternatively, test yeasts are cultured and a yeast with a lower haze-producing capability is selected, thereby selecting a yeast suitable for brewing desired alcoholic beverages.

In these cases, the test yeasts or the reference yeast may be, for example, a yeast introduced with the vector of the invention, a yeast in which an expression of a polynucleotide (DNA) of the invention has been increased, a yeast in which an expression of a protein of the invention has been increased, an artificially mutated yeast or a naturally mutated yeast The production amount of haze can be determined by, for example, the methods described in P. W. Gales et al.: J. Am. Soc. Brew. Chem. 58, 101-107 (2000) . The mutation treatment may employ any methods including, for example, physical methods such as ultraviolet irradiation and radiation irradiation, and chemical methods associated with treatments with drugs such as EMS (ethylmethane sulphonate) and N-methyl-N-nitrosoguanidine (see, eg, Yasuji Oshima Ed, biochemistry experiments vol. 39, Yeast Molecular Genetic Experiments, pp. 67-75, JSSP).

In addition, examples of yeasts used as the reference yeast or the test yeasts include any yeasts that can be used for brewing, for example, brewery yeasts for beer, wine, sake and the like. More specifically, yeasts such as genus Saccharomyces may be used (e.g, S pastorianus, S. cerevisiae, and S. carlsbergensis). According to the present invention, a lager brewing yeast, for example, Saccharomyces pastorianus W34/70; Saccharomyces carlsbergensis NCYC453 or NGYC456; or Saccharomyces cerevisiae NBRC-1951,NBRC1952, NBRC1953 or NBRC1954 may be used. Further, whiskey yeasts such as Saccharomyces cerevisiae NCY90, wine yeasts such as wine yeasts #1, 3 and 4 from the Brewing Society of Japan; and sake yeasts, such as sake yeast #7 and 9 from the Brewing Society of Japan may also be used but not limited thereto. In the present invention, lager brewing yeasts such as Saccharomyces pastorianus may preferably be used. The reference yeast and the test yeasts may be selected from the above yeasts in any combination.

### EXAMPLES

Hereinafter, the present invention will be described in more detail with reference to working examples. The present invention, however, is not limited to the examples described below.

### Example 1

Cloning of Gene Encoding a Cell Wall Mannoprotein (non-ScCWP2)

A specific novel gene encoding p. cell wall mannoprotein (non-ScCWP2) gene (SEQ ED NO: 1) from a lager brewing yeast were found, as a result of a search utilizing the comparison database described in Japanese Patent Application Laid-Open No. 2004-283169. Based on the acquired nucleotide sequence information, primers non-ScCWP2_for (SEQ ID NQ: 5) and non-ScCWP2_rv (SEQ ID NO: 6) were designed to amplify the nonScCWP2 full-length genes. PCR was carried out using chromosomal DNA of a genome sequencing strain, Saccharomyces pastorianus Weihenstephan 34/70 strain (which may be abbreviated to “W34/70 strain”), as a template to obtain DNA fragments (about 0.3 kb) including the full-length gene of non-ScMET17.

The thus-obtained non-ScCWP2 gene fragment was inserted into pCR2.1-TOPO vector (Invitrogen) by TA cloning. The nucleotide sequences of non-ScCWP2 gene were analyzed according to Sanger's method (F. Sanger, Science, 214: 1215, 1981) to confirm the nucleotide sequence.

### Example 2

Analysis of Expression of non-ScCWP2 Gene during Beer Fermentation

A beer fermentatioa test was conducted using a lager brewing yeast, Saccharomyces pastorianus Wemenstephari 34/70 strain and then mRNA extracted from yeast cells during fermentation was analyzed by a DNA microarray.

| | |
|---|---|
| Wort extract concentration | 12.69% |
| Wort content | 70 L |
| Wort dissolved oxygen concentration | 8.6 ppm |
| Fermentation temperature | −15° C. |
| Yeast pitching rate | 12.8 × 10⁶ cells/mL |

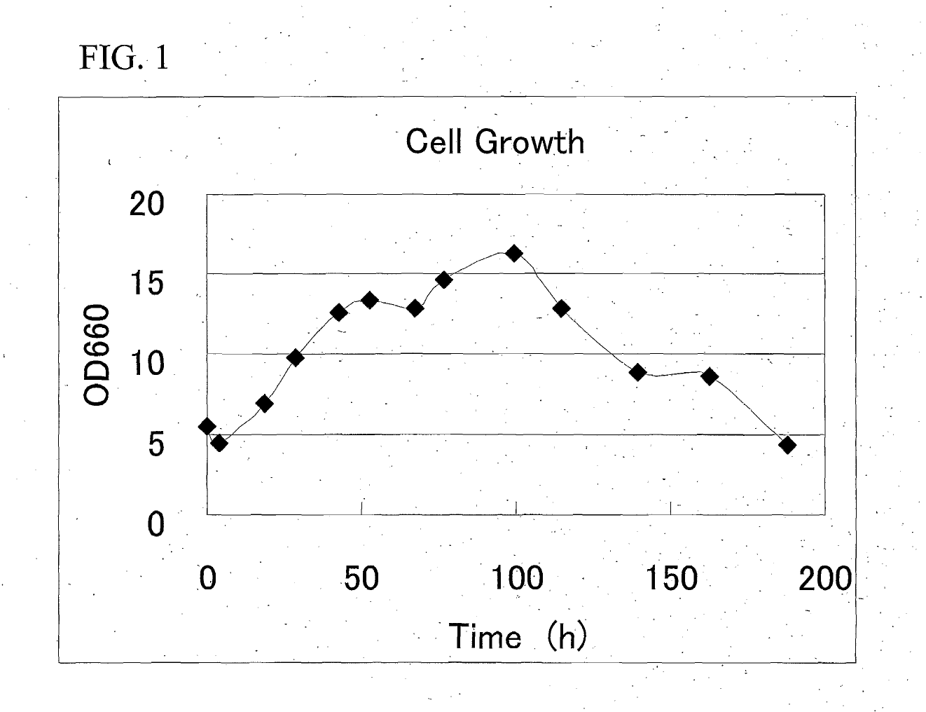

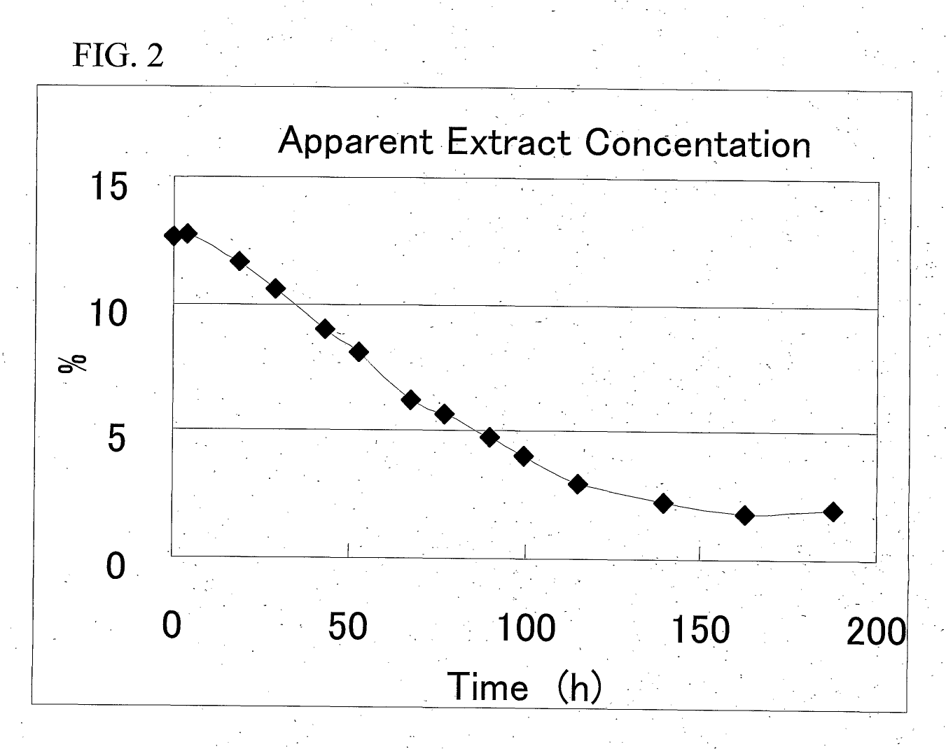

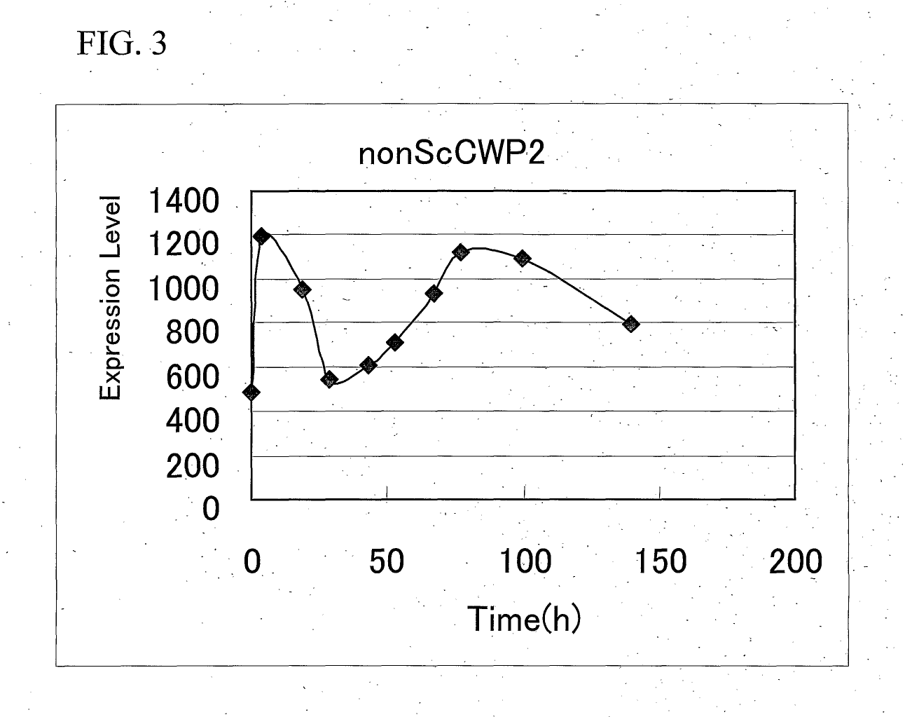

Sampling, of fermentation liquor was performed with time, and variation with time of yeast growth amount (FIG. 1) and apparent extract concentration (FIG. 2) was observed. Simultaneously, sampling of yeast cells was performed, and the prepared mRNA was subjected to be biotin-labeled and was hybridized to a beer yeast DNA microarray. The signal was detected using GCOS; GeneChip Operating Software 1.0 (manufactured by Affymetrix Co.). Expression pattern of non-ScCWP2 gene is shown in FIG. 3. As a result, it was confirmed that nori-ScCWP2 gene was expressed in the general beer fermentation.

### Example 3

High Expression of non-ScCWP2 Gene

The non-ScCWP2/pCR2.1-TOPO described in Example 1 was digested using the restriction enzymes Sad and NotI, so as to prepare a DNA fragment containing the entire length of the protein-encoding region. This fragment was ligated to pYCGPYNot treated with the restriction enzymes SacI and NotI, thereby constructing the non-ScCWP2 high expression vector non-ScCWP2/pYCGPYNot. pYCGPYNot is the YCp-type yeast expression vector. The inserted gene is highly expressed by the pyruvate kinase gene PYK1 promoter. The geneticin-resistant gene G418^{r} is included as the selection marker in the yeast, and the ampicillin-resistant gene Amp^{r} is included as the selection marker in Escherichia coli.

Using the non-ScCWP2 high expression vector prepared by the above method, the strain Saccharomyces pasteurianus Weihenstephaner 34/70 was transformed by the method described in Japanese Patent Application Laid-open No. H7-303475. The transformant was selected in a YPD plate culture (1% yeast extract, 2% polypeptone, 2% glucose, 2% agar) containing 300 mg/L of geneticin.

### Example 4

### Analysis of Amount of Haze Produced in Test Brewing of Beer

A fermentation test was carried out under the following conditions using the parent strain and the non-ScCWP2 highly expressed strain obtained in Example 3.

| | |
|---|---|
| Wort extract concentration | 11.85% |
| Wort content | 2 L |
| Wort dissolved oxygen concentration | approx. 8 ppm |
| Fermentation temperature | 15° C. (fixed) |
| Yeast pitching rate | 10 g wet yeast cells/2 L of wort |

The fermentation broth was sampled over time, and the change over time in the yeast growth rate (OD660) and me amount of extract consumed were determined. For quantitative determination of the haze in the broth, floating yeast was precipitated by centrifugation of the broth at 5,000 rpm for 10 minutes, the supernatant was retrieved and was filtered with diatomaceous earth, and the filtrate was used for measuring haze. The above-described sample was filtered using diatomaceous earth placed on a metal mesh with a pore size of 50 μm. After filtration, the filtrate was maintained on ice-water (0° c. ) for 24 hours for facilitating the appearance of haze. The level of ‘’ haze of the sample was measured using a haze meter (trade name: Siglist electrophotometer, manufactured by Siglist Co.), and the measured value was used as T-haze (total turbidity). The value measured by solubilizing chilled coagulants at 28° C. was used as P-Haze (permanent turbidity), and the difference between T-Haze and P-Haze was used as haze value by the chilled coagulant, or C-Haze (turbidity by chilled coagulant). The haze value is expressed in Helm unit (1 Helm=0.1 FTU (Formazin Turbidity Unit): Document; P. W. Gales et al., J. Am. Soc. Brew. Chem., 58, 101:107, 2000). The results obtained are shown in Table 1.

TABLE 1

| | T-Haze | | P-Haze | | C-Haze | |
|---|---|---|---|---|---|---|
| | Measured | Av- | Measured | | Measured | Av- |
| | value | erage | value | Average | value | erage |
| nonScCWP2 | 39 | 41 | 30 | 31 | 9 | 10 |
| highly | 42 | | 32 | | 10 | |
| expressed | | | | | | |
| strain | | | | | | |
| Parent strain | 65 | 64 | 48 | 46 | 17 | 18 |
| | 62 | | 44 | | 18 | |

From Table 1, the amount of T-Haze that had been produced on the completion of fermentation was 64 Helm for the parent strain, whereas it was 41 Helm for the nonScCWP2 highly expressed strain. For the amount of P-Haze, it was 46 Helm for the parent strain, whereas it was 31 Helm for the nonScC WP2 highly expressed strain. For the amount of C-Haze, it was 18 Helm for the parent strain, whereas it was 10 Helm for the nonScCWP2 highly expressed strain. It is clear from these results that the amount of Haze produced was reduced about 34-45% with high expression of the non-ScCWP2 gene.

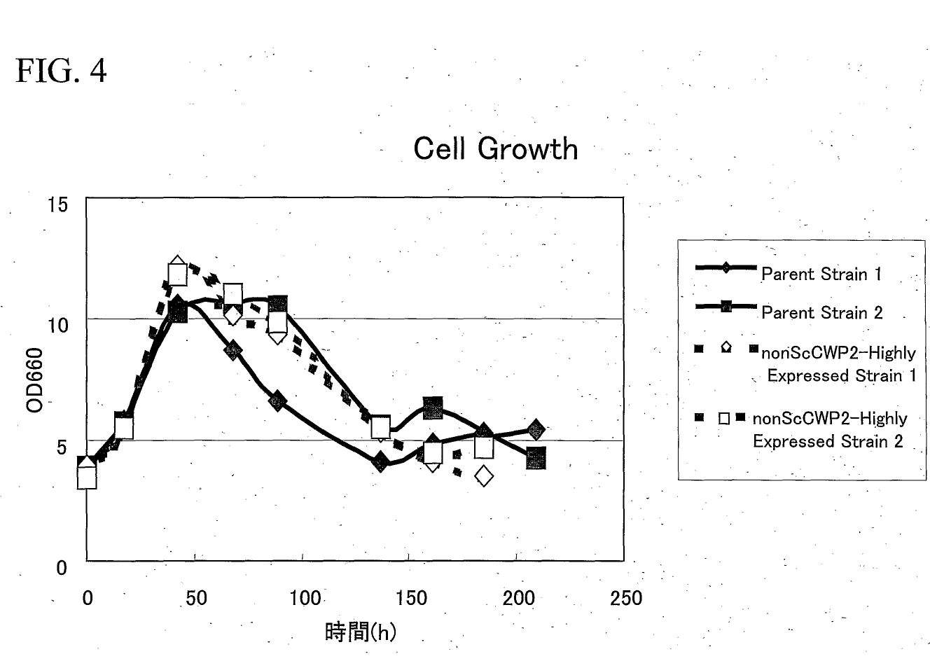

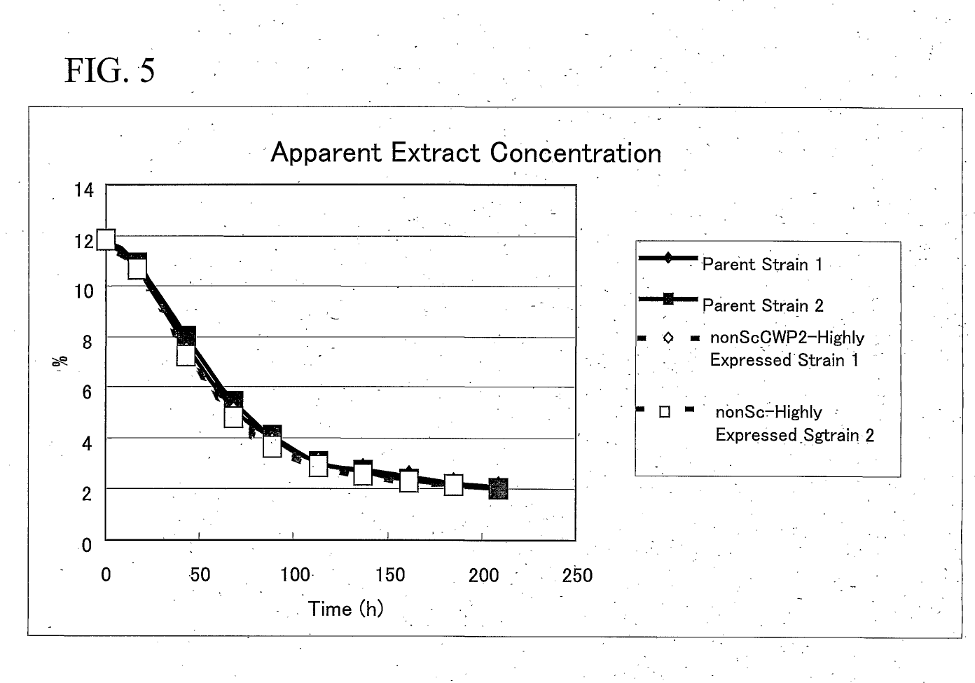

In addition, the results obtained in fee working examples are shown in FIGS. 4 and 5. FIG. 4 shows the cell growth with time upon fermentation test in this Example. The horizontal axis represents fermentation time while the vertical axis represents optical density at 660 nm (OD660). FIG. 5 shows fee sugar consumption wife time upon beer fermentation test in this Example. The horizontal axis represents fermentation time while the vertical axis represents apparent extract concentration (w/w %).

### Example 5

### Cloning of Cell Wall Mannoprotein-Encoding Gene (ScCWP2)

Primers ScCWP2_for (SEQ ID NO: 7) and ScCWP2_rv (SEQ ID NO: 8) were designed to amplify the ScCWP2 full-length genes. PCR was carried out using chromosomal DNA of S. cerevisiae X2180-1A, as a template to obtain DNA fragments (about 0.3 kb) including the full-length gene of ScCWP2.

The thus-obtained ScCWP2 gene fragments were inserted into pCR2.1-TOPO vector (Invitrogen) by TA cloning,, respectively. The nucleotide sequences of ScCWP2 gene were analyzed according to Sanger's method (F. Sanger, Science, 214: 1215, 1981) to confirm the nucleotide sequence.

### Example 6

Analysis of Expression of ScCWP2 Gene during Beer Fermentation

A fermentation test was conducted using a lager brewing yeast, Saccharomyces pastorianus W34/70 strain and then mRNA extracted from yeast cells during fermentation was analyzed by a DNA microarray.

| | |
|---|---|
| Wort extract concentration | 12.69% |
| Wort content | 70 L |
| Wort dissolved oxygen concentration | 8.6 ppm |
| Fermentation temperature | 15° C. |
| Yeast pitching rate | 12.8 × 10⁶ cells/mL |

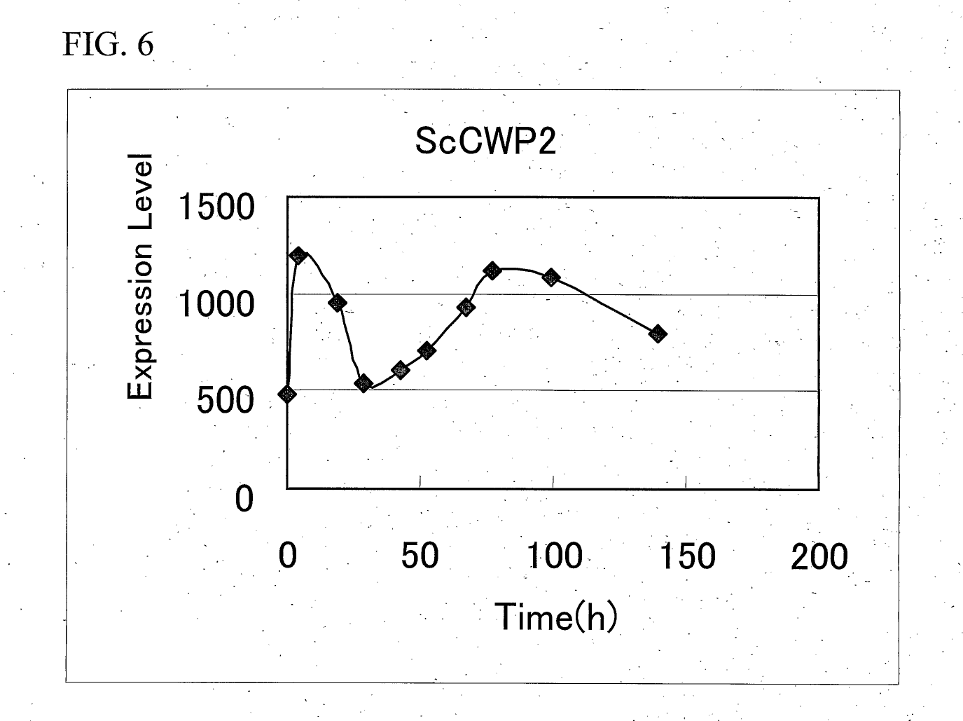

Sampling of fermentation liquor was performed with time, and variation with time of fee cell growth (FIG. 4) and apparent extract concentration (FIG. 5) were observed. Simultaneously, yeast cells were sampled to prepare mRNA, and the prepared mRNA was labeled with biotin and was hybridized to a beer yeast DNA microarray. The signal was detected using GCOS; GeneGhip Operating Software 1.0 (manufactured by Affymetrix Co.). Expression pattern of ScCWP2 gene is shown in FIG. 6. As a result, it was confirmed that ScCWP2 gene was expressed in die general beer fermentation.

### Example 7

### Preparation of ScCWP2-Higly Expressed Gene

The plasmid TOPO/ScCWP2 described in Example 5 was treated, with the restriction enzymes XhoI and BamHI so as to prepare about 0.7 kb DNA fragment containing the ScCWP2 gene. This fragment was ligated to pUP3GLP2 treated with the restriction enzymes XhoI and BamHI, thereby constructing the ScCWP2 high expression vector pUP-ScCWP2. A yeast expression vector pUP3GLP2 is a vector of Yip type (chromosome integrated type) containing orotidine-5′phosphate decarboxylase gene URA3 as a homologous recombination site. The inserted gene is highly expressed by the glycerddehyde-3′-phosphate dehydrogenase gene TDH3 promoter/terminator. A drug-resistant gene YAP1 is incorporated as the selection marker in tile yeast under the control of promoter/terminator of galactokinase gene GAL1, and the expression is induced in a medium containing galactose. The ampicillin-resistant gene. Amp^{r} is included as the selection marker in Escherichia coli.

Using the nonScCWP2 high expression vector prepared by the above method, the strain Weihenstephan Nr. 164 was transformed by the method described in Japanese Patent Application Laid-open No. H7-303475. The cerulenin-resistant strain was selected in a YPGal plate culture (1% yeast extract, 2% polypeptohe, 2% galactose, 2% agar) containing 1.0 mg/L of cerulenin.

### Example 8

### Analysis of Amount of Haze Produced in Beer Brewing Testing

A fermentation test was carried out under the following conditions using the parent strain and the ScCWP2-highly expressed strains obtained in Example 7.

| | |
|---|---|
| Wort extract concentration | 11.85% |
| Wort content | 2 L |
| Wort dissolved oxygen concentration | approx. 8 ppm |
| Fermentation temperature | 15° C. (fixed) |
| Yeast pitching rate | 10 g wet yeast cells/2 L of wort |

The fermentation broth was sampled over time, and the change over time in the yeast growth rate (OD660) and-the amount of extract consumed were determined. For quantitative determination of the haze in the broth, floating yeast was precipitated by centrifugation of the broth at 5,000 rpm for 10 minutes, the supernatant was retrieved and was filtered with diatomaceous earth, and the filtrate was used for measuring haze. The above-described sample was filtered using diatomaceous earth placed on a metal mesh with a pore size of 50 μm. After filtration, the filtrate was maintained on ice-water (0° C.) for 24 hours for facilitating the appearance of haze. The level of haze of the sample was measured using a haze meter (trade name: Siglist electrophotometer, manufactured by Siglist Co.), and the measured value was used as T-haze (total turbidity). The value measured by solubilizing chilled coagulants at 28° C. was used as P-Haze (permanent turbidity), and the difference between T-Haze and P-Haze was used as haze value by the chilled coagulant, or C-Haze (turbidity by chilled coagulant). The haze value is expressed in Helm unit (1 Helm=0.1 FTU (Formazin Turbidity Unit): Document; P. W. Gales et al., J. Am, Soc. Brew. Chem., 58, 101-107, 2000). The results obtained are shown in Table 2.

TABLE 2

| | T-Haze | | P-Haze | | C-Haze | |
|---|---|---|---|---|---|---|
| | Measured | | Measured | | Measured | Av- |
| | value | Average | value | Average | value | erage |
| Parent | 32 | 33 | 23 | 23 | 9 | 10 |
| strain | 33 | | 23 | | 10 | |
| ScCWP2 | 21 | 24 | 16 | 18 | 5 | 7 |
| highly | 27 | | 19 | | 8 | |
| expressed | | | | | | |
| strain | | | | | | |

From Table 2, the amount of T-Haze that had been produced on completion of fermentation was 33 Helm for the parent strain, whereas it was 24 Helm for the ScCWP2 highly expressed strains. For the amount of P-Haze, it was 23 Helm for the parent strain, whereas it was 18 Helm for the ScCWP2 highly expressed strain. For the amount of C-Haze, it was 10 Helm for the parent, strain, whereas it was 7 Helm for me ScCWP2 highly expressed strain. It was clear from these results that the amount of Haze production was reduced about 22-33% by high expression of the ScCWP2 gene. No substantial difference was observed in the proliferation rate and malt extract consumption rate between the parent strain and are transformed strain.

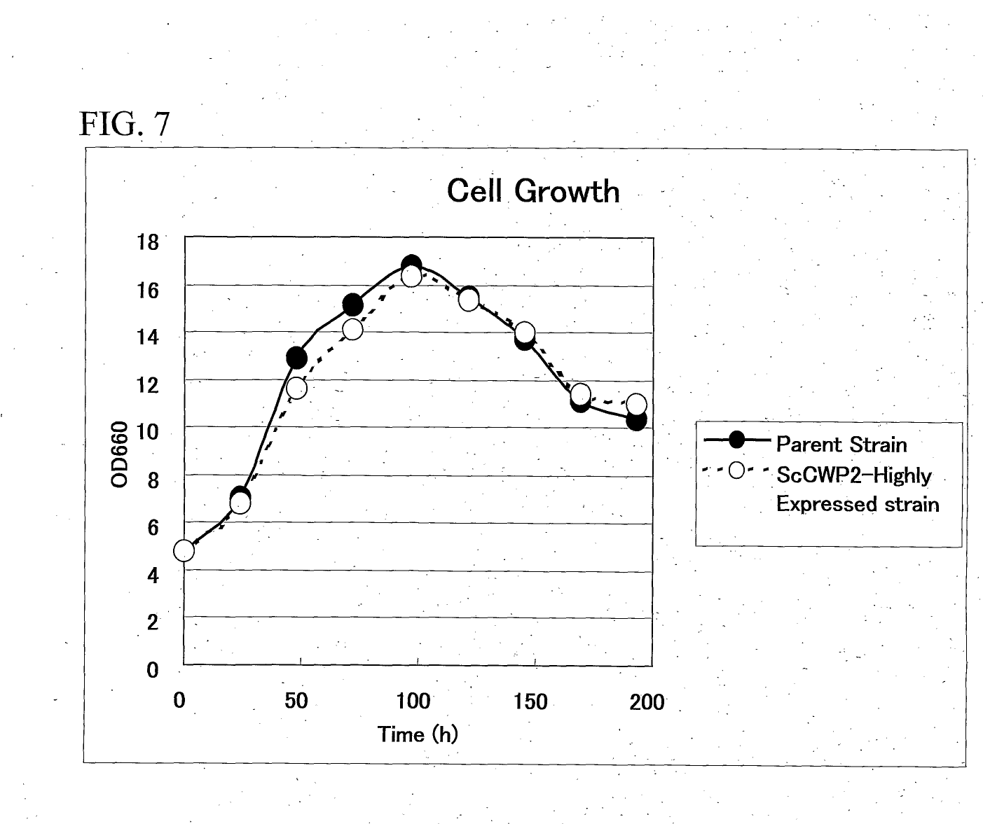

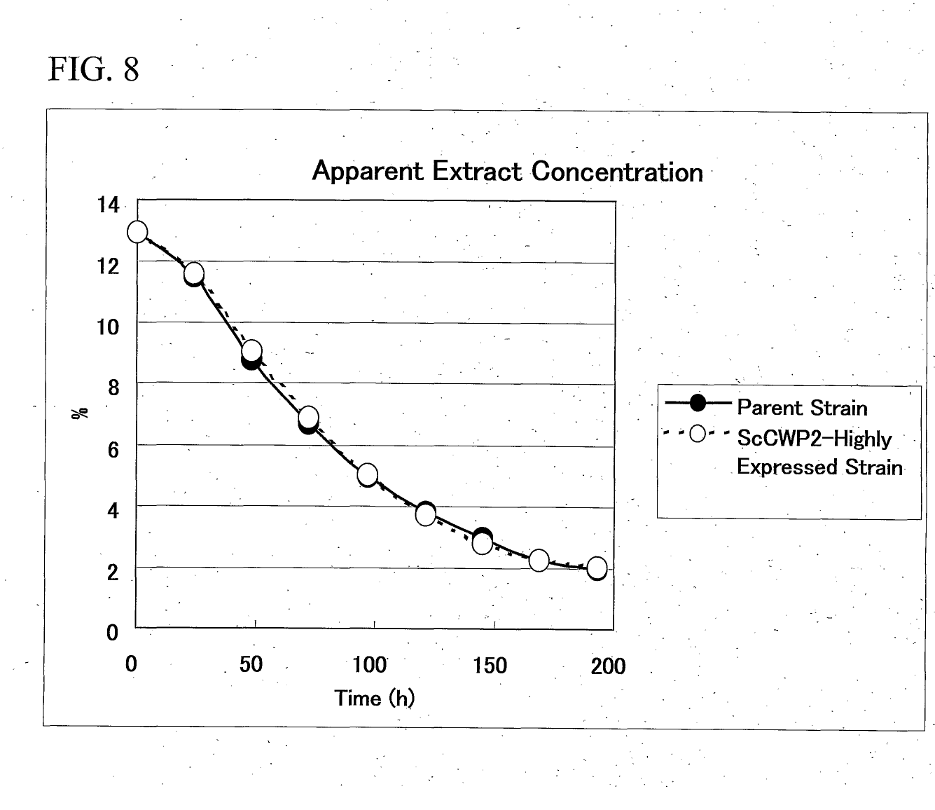

In addition, the results obtained in the working examples are shown in FIGS. 7 and 8. FIG. 7 shows the cell growth with time upon fermentation test in this Example. The horizontal axis represents fermentation time while the vertical axis represents optical density at 660 am (OD660). FIG. 8 shows the sugar consumption with time upon beer fermentation test in this Example. The horizontal axis represents fermentation time while the vertical axis represents apparent extract concentration (w/w %).

### INDUSTRIAL APPLICABILITY

According to the method of producing alcoholic beverages of the present invention, since the amount of haze can be lowered in beer fermentation and the finished product, alcoholic beverages having low amount of haze can be produced.

1. A polynucleotide selected from the group consisting of:
(a) a polynucleotide comprising a polynucleotide consisting of the nucleotide sequence of SEQ ID NO: 1;
(b) a polynucleotide comprising a polynucleotide encoding a protein consisting of the amino acid sequence of SEQ ID NO: 2;
(c) a polynucleotide comprising a polynucleotide encoding a protein consisting of the amino acid sequence of SEQ ID NO: 2 with one or more amino acids thereof being deleted, substituted, inserted and/or added, and functioning as a cell wall mannoprotein;
(d) a polynucleotide comprising a polynucleotide encoding a protein having an amino acid sequence having 60% or higher identity with the amino acid sequence of SEQ ID NO: 2, and functioning as a cell wall mannoprotein;
(e) a polynucleotide comprising a polynucleotide which hybridizes to a polynucleotide consisting of a nucleotide sequence complementary to the nucleotide sequence of SEQ ID NO: 1 under stringent conditions, and which encodes a protein functioning as a cell wall mannoprotein; and
(f) a polynucleotide comprising a polynucleotide which hybridizes to a polynucleotide consisting of a nucleotide sequence complementary to the nucleotide sequence of the polynucleotide encoding the protein of the amino acid sequence of SEQ ID NO: 2 under stringent conditions, and which encodes a protein functioning as a cell wall mannoprotein.

2. The polynucleotide of claim 1 selected from the group consisting of:
(g) a polynucleotide comprising a polynucleotide encoding a protein consisting of the amino acid sequence of SEQ ID NO: 2, or encoding an amino acid sequence of SEQ ID NO: 2 wherein 1 to 10 amino acids thereof is deleted, substituted, inserted, and/or added, and wherein said protein functions as a cell wall mannoprotein;
(h) a polynucleotide comprising a polynucleotide encoding a protein having 90% or higher identity with the amino acid sequence of SEQ ID NO: 2, and functioning as a cell wall mannoprotein; and
(i) a polynucleotide comprising a polynucleotide which hybridizes to SEQ ID NO: 1 or which hybridizes to a nucleotide sequence complementary to the nucleotide sequence of SEQ ID NO: 1 under stringent conditions, and which encodes a protein functioning as a cell wall mannoprotein.

3. The polynucleotide of claim 1 comprising a polynucleotide consisting of SEQ ID NO: 1.

4. The polynucleotide of claim 1 comprising a polynucleotide encoding a protein consisting of SEQ ID NO: 2.

5. The polynucleotide of claim 1, wherein the polynucleotide is DNA.

6. A protein encoded by the polynucleotide of claim 1.

7. A vector comprising the polynucleotide of claim 1.

8. A vector comprising the polynucleotide selected from the group consisting of:
(j) a polynucleotide comprising a polynucleotide encoding a protein consisting of the amino acid sequence of SEQ ID NO: 4, or encoding an amino acid sequence of SEQ ID NO: 4 wherein 1 to 10 amino acids thereof is deleted, substituted, inserted, and/or added, and wherein said protein functions as a cell wall mannoprotein;
(k) a polynucleotide comprising a polynucleotide encoding a protein having 90% or higher identity with the amino acid sequence of SEQ ID NO: 4, and functioning as a cell wall mannoprotein; and
(l) a polynucleotide comprising a polynucleotide which hybridizes to SEQ ID NO: 3 or which hybridizes to a nucleotide sequence complementary to the nucleotide sequence of SEQ ID NO: 3 under high stringent conditions, and which encodes a protein functioning as a cell wall mannoprotein.

9. A yeast comprising the vector of claim 7.

10. The yeast of claim 9, wherein a haze-producing ability is reduced by introducing the vector of comprising the polynucleotide.

11. The yeast of claim 10, wherein a haze-producing ability is reduced by increasing an expression level of the protein encoded by the polynucleotide.

12. A method for producing an alcoholic beverage comprising culturing the yeast of claim 9.

13. The method for producing an alcoholic beverage of claim 12, wherein the brewed alcoholic beverage is a malt beverage.

14. The method for producing an alcoholic beverage of claim 12, wherein the brewed alcoholic beverage is wine.

15. An alcoholic beverage produced by the method of claim 12.

16. A method for assessing a test yeast for its haze-producing capability, comprising using a primer or a probe designed based on a nucleotide sequence of a gene encoding a cell wall mannoprotein having the nucleotide sequence of SEQ ID NO: 1 or SEQ ID NO: 3.

17. A method for assessing a test yeast for its haze-producing capability, comprising: culturing a test yeast; and measuring an expression level of a gene encoding a cell wall mannoprotein having the nucleotide sequence of SEQ ID NO: 1 or SEQ ID NO: 3.

18. A method for selecting a yeast, comprising: culturing test yeasts; quantifying the protein according to Claim 6 or measuring an expression level of a gene encoding a cell wall mannoprotein having the nucleotide sequence of SEQ ID NO: 1 or SEQ ID NO: 3; and selecting a test yeast having said protein amount or said gene expression level according to a target haze-producing capability.

19. The method for selecting a yeast according to claim 18, comprising: culturing a reference yeast and test yeasts; measuring an expression level of a gene encoding a cell wall mannoprotein having the nucleotide sequence of SEQ ID NO: 1 or SEQ ID NO: 3 in each yeast; and selecting a test yeast having the gene expressed higher than that in the reference yeast.

20. The method for selecting a yeast according to claim 18, comprising: culturing a reference yeast and test yeasts; quantifying the protein encoded by the polynucleotide in each yeast; and selecting a test yeast having said protein for a larger amount than that in the reference yeast.

21. A method for producing an alcoholic beverage comprising:
conducting fermentation for producing an alcoholic beverage using the yeast according to claim 9 or a yeast selected by the method according to claim 18; and adjusting the production amount of haze.
